(19) United States
(12) Patent Application Publication
(10) Pub. No.: US 2023/0404546 A1
(43) Pub. Date: Dec. 21, 2023

(54) BODILY FLUID INDICATOR DEVICES AND METHODS

(71) Applicant: Fishburne May, LLC, Chapel Hill, NC (US)

(72) Inventors: Rita Marie May, Chapel Hill, NC (US); Clifton M. Carey, Pittsburgh, PA (US); Coty Fishburne, JR., Rock Hill, SC (US)

(73) Assignee: Fishburne May, LLC, Chapel Hill, NC (US)

(21) Appl. No.: 18/240,083

(22) Filed: Aug. 30, 2023

Related U.S. Application Data
(63) Continuation-in-part of application No. 17/171,196, filed on Feb. 9, 2021.
Application No. 17/171,196 is a continuation of application No. 16/050,531, filed on Jul. 31, 2018, now Pat. No. 10,912,540.
Application No. 16/050,531 is a continuation of application No. 15/439,134, filed on Feb. 22, 2017, now Pat. No. 10,058,307.
(60) Provisional application No. 62/395,683, filed on Sep. 16, 2016.
(60) Provisional application No. 62/298,561, filed on Feb. 23, 2016.

(51) Int. Cl. A61B 10/00; A61B 5/145; A61B 5/1473; A61B 5/00

(52) U.S. Cl. CPC A61B 10/0051; A61B 5/14507; A61B 5/14539; A61B 5/1473; A61B 5/4261; A61B 2010/0003; A61B 2010/0006; A61B 2010/0074; A61B 2562/0295; A61B 5/14546; A61B 5/01

(57) ABSTRACT

Bodily fluid indicator devices, systems, and methods are provided. Such devices, systems, and methods can effectively indicate whether the amount of bodily fluid being produced is within a measurable range, or can characterize various components or features of a bodily fluid. In an exemplary embodiment, a bodily fluid indicator device includes a substrate having a surface. Some portions of the surface are covered with a region of absorbent material and other portions of the surface are devoid of the absorbent material. A graduated scale or a reactive feature can be printed on the absorbent material. The device is configured to indicate an amount and/or a composition a bodily fluid present on an area of a patient or subject.

### CROSS-REFERENCE TO RELATED APPLICATIONS

This application is a continuation-in-part of U.S. patent application Ser. No. 17/171,196 filed Feb. 9, 2021, which is a continuation of U.S. patent application Ser. No. 16/050,531 filed Jul. 31, 2018 (issued as U.S. Pat. No. 10,912,540 on Feb. 9, 2021), which is a continuation of U.S. patent application Ser. No. 15/439,134 filed Feb. 22, 2017 (issued as U.S. Pat. No. 10,058,307 on Aug. 28, 2018), which claims the priority benefit under 35 U.S.C. § 119(e) of U.S. Provisional Patent Application No. 62/298,561 filed on Feb. 23, 2016 and U.S. Provisional Patent Application No. 62/395,683 filed on Sep. 16, 2016, the contents of each of which are herein incorporated by reference in their entirety.

### FIELD

The technology described herein generally relates to devices and methods for obtaining bodily fluid of a subject and, in particular, to bodily fluid indicator devices and methods for collecting, indicating, and characterizing various attributes associated with the bodily fluid.

### BACKGROUND

The human or mammalian body produces and secretes various bodily fluids, including, but not limited to saliva, sweat, nasal fluid, vaginal fluid, etc. Changes in the amount of a bodily fluid being produced and/or changes in the composition of the bodily fluid being produced can be indicators of a subject's overall health. For example, a shortage or overage in production of some types of bodily fluids can be indicative of disease or a health concern, such as the existence of a tumor, an autoimmune disease, or diabetes, just to name a few.

Currently, tests that indicate the amount and/or composition of bodily fluid being produced are determined using measures of fluid (e.g., saliva is removed from the mouth and then measured). Existing tests are also performed over a period of several minutes, typically five minutes or longer. The prolonged length of these tests can be uncomfortable to the subject. Accordingly, a need exists for improved bodily fluid indicator devices, methods, and systems which overcome the deficiencies of conventional devices and methods and can easily and effectively indicate whether the amount of bodily fluid being produced is within a measurable range, or furthermore can characterize bodily fluid samples collected.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Embodiments of the technology described herein are directed towards bodily fluid indicator devices, systems, and methods that can be configured to collect and/or characterize bodily fluid samples of a subject, for instance from a human or an animal. Bodily fluid indicator devices and methods are provided and disclosed herein. In an exemplary embodiment, a bodily fluid indicator device is provided. The device comprises a surface and a graduated scale or reactive feature. Some portions of the surface are covered with a region of absorbent material and other portions of the surface are devoid of the absorbent material. The graduated scale or reactive feature can be printed on the absorbent material. The graduated scale can be printed using a U.S. Food and Drug Agency (FDA) approved ink, including but not limited to a food grade ink.

In further embodiments, the substrate is flexible. The substrate may comprise an organic or inorganic material. The substrate can comprise paper and be planar or curved.

Moreover, a device can comprise a plurality of bendable tabs that extend from the surface. The tabs are configured to rest against a portion of a subject's body during a bodily fluid measurement.

Additionally, the substrate can comprise a body portion and a stem portion extending from the body portion. The body portion can be at least 0.5 inches wide and the stem portion can be at least 0.125 inches wide. Notably, the devices set forth herein are configured to indicate an amount and/or a composition a bodily fluid present on an area of a patient or subject. The bodily fluid can comprise saliva, sweat, nasal fluid, breast milk, breast discharge, or vaginal fluid.

In a further aspect, a method of using a bodily fluid indicator device is provided. The method can comprise providing a bodily fluid indicator device comprising a region of absorbent material and a graduated scale printed thereon. The method can further comprise contacting a bodily fluid with the indicator device for a period of time sufficient to cause the bodily fluid to migrate over a portion of the graduated scale, wherein the period of time is less than one minute. The method can further comprise determining a measurable amount of bodily fluid being produced based on a proportion of the graduated scale over which the bodily fluid has migrated.

In yet a further aspect, an additional method of using a bodily fluid indicator device is provided. The method comprises providing a bodily fluid indicator device comprising a region of absorbent material and one or more reactive features printed thereon and contacting a bodily fluid with the indicator device for a period of time sufficient to cause the bodily fluid to migrate over a portion of the one or more reactive features. The period of time is less than one minute. The method can further comprise indicating an absence or presence of a chemical constituent forming the bodily fluid based on a reaction of the one or more reactive features over which the bodily fluid has migrated. The reaction can comprise a change in color, intensity, visibility, size, and/or shape of the one or more reactive features

These and other embodiments are described in more detail in the detailed description which follows. Additional objects, advantages, and novel features of the invention will be set forth in part in the description which follows, and in part will become apparent to those skilled in the art upon examination of the following, or can be learned by practice of the invention.

### BRIEF DESCRIPTION OF THE DRAWINGS

Aspects of the technology presented herein are described in detail below with reference to the accompanying drawing figures, the components and/or features of which are not necessarily drawn to scale, wherein:

FIGS. 1A-IF are various views of a bodily fluid indicator device, according to some embodiments;

FIGS. 2A-2B are schematic views of various bodily fluid indicator devices, according to some embodiments;

FIG. 3 is a flow chart of an example method for using a bodily fluid indicator device, according to some embodiments;

FIGS. 4A-4C are schematic views illustrating aspects of a bodily fluid indicator device, in accordance with some aspects of the technology described herein;

FIG. 5 is a diagram of an example bodily fluid indicator device, in accordance with some aspects of the technology described herein;

FIG. 6A is an example system of a bodily fluid indicator device and reader, in accordance with some aspects of the technology described herein;

FIG. 6B is an example system of a bodily fluid indicator device and reader, in accordance with some aspects of the technology described herein;

FIG. 7A is an example body fluid indicator device, in accordance with some aspects of the technology described herein; and

FIG. 7B is an example body fluid indicator device, in accordance with some aspects of the technology described herein.

### DETAILED DESCRIPTION

The subject matter of aspects of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps disclosed herein unless and except when the order of individual steps is explicitly described.

Embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

Additionally, in any disclosed embodiment, the terms “substantially,” “approximately,” and “about” may be substituted with “within [a percentage] of” what is specified, where the percentage includes 0.1, 1, 5, and 10 percent.

Bodily fluid indicator devices and methods are described herein which may provide one or more advantages over prior devices. Bodily fluid devices and methods herein are configured to indicate whether the type and/or amount of bodily fluid being produced is measurable. Bodily fluid devices and methods herein are also configured to indicate other attributes associated with a bodily fluid, such as attributes relating to composition (e.g., specific elements or chemical present, whether a yeast or bacteria are present, whether a hormone is present, etc.), temperature, or pH. Notably, devices and methods described provide quicker and/or more easily readable results than prior or conventional devices.

For example, the devices and methods herein are configured to test bodily fluid via a bodily fluid indicating device to indicate whether the chemical composition and/or amount of bodily fluid is within an accepted range, and the test can be performed in less than one minute.

Moreover, devices and/or methods described herein can be easily obtained and/or used by any health care provider (e.g., dentist, physician, nurse, pharmacist, etc.) or a subject, either in a clinical setting, a pharmacy setting, or in a home-use environment (i.e., a self-test). The self-test can be performed over a short course of time for an indication of whether the bodily fluid production is increasing or decreasing.

### I. Bodily Fluid Indicator Devices

In some aspects, devices for indicating the type and/or amount of bodily fluid being produced are provided. In some embodiments, devices herein comprise a substrate. The substrate can be rigid, semi-rigid, or flexible. In some embodiments, the device comprises a thin device having a thin substrate, the device being configured for insertion within and/or contacting a subject's body (e.g., a mouth, eye, or nipple) to indicate an amount of bodily fluid present, a pH or temperature of the fluid, a composition forming the bodily fluid, and/or compositional elements (e.g., chemicals, bacterial, hormones, yeast, etc.) present or absent in the bodily fluid. In some embodiments, the device comprises a multidimensional solid or hollow device configured for insertion within the subject's body (e.g., a vagina or nose) for indicating an amount of bodily fluid present and/or a composition forming the bodily fluid.

The device substrate can comprise at least one surface that faces a source of the bodily fluid (e.g., a tongue, a vaginal wall, a nasal cavity, etc.). Some portions of the surface can be covered with a region or area of absorbent material and other portions of the surface can be devoid of the absorbent material. The absorbent material can comprise a porous material, a fibrous material, a porous paper, a filter paper, an absorbent paper, a woven or non-woven material, etc. Bodily fluids can advance or migrate from the fluid source (e.g., a tongue, vaginal wall, etc., or portion thereof), which contacts a proximal end of the absorbent material, towards a distal end of the absorbent material. The distal end of the absorbent material can be disposed outside of the subject's body. Thus, the fluid can migrate via capillary or osmotic action from one end of the absorbent material that is inside the subject's body towards a second end of the absorbent material that is outside of the subject's body, for example, where a graduated scale or one or more features are printed.

In certain embodiments, the substrate can comprise or be formed from a polymeric material such as a polystyrene (PS) (e.g. 0.01 polystyrene, 0.03 polystyrene, or 0.04 polystyrene). However, it is to be understood that the substrate is not limited to a polystyrene. Other materials may include polymeric materials, organic materials, plastic, polyethylene terephthalate (PET), polyethylene (PE), high-density polyethylene (HDPE), polyvinylidene chloride (PVDC), polypropylene (PP), high impact polystyrene (HIPS), polyamide (PA), and/or polycarbonate (PC). Non-plastic materials may also be used. In some embodiments, the substrate comprises or is formed from a food approved or food safe material, and/or comprises or is formed from a material which minimizes or reduces the risk of injury to the subject's mouth, gums, and/or teeth.

The substrate can have any dimensions or shape not inconsistent with the objectives of the present invention. For example, in some embodiments, the substrate is square, rectangular, and/or comprises one or more curved sections. Such shapes may be adapted as a “one size fits all” shape or size so that subjects of most or all ages or sizes may use the devices without modification. In addition, the substrate's size and/or shape may accommodate a subject with all of his or her teeth, some of his or her teeth missing, or with no teeth.

In some embodiments, a graduated scale and/or one or more reactive features may be printed on or over the absorbent material. The phrase “reactive feature” refers to a feature printed on the test device that has a chemical composition which is reactive to the presence or absence of a chemical, bacteria, yeast, temperature, pH, or a specific Biomarker. The chemical composition may react by changing color, intensity, size, shape, visibility, etc., in reaction to detecting the presence or absence of the chemical, bacteria, yeast, etc. For example, and in some embodiments, where specific bacteria are present in the bodily fluid and a reactive feature which targets the specific bacteria can be printed on the device, the feature may appear (i.e., become visible), change color, size, intensity, etc., to indicate that the specific bacteria are present.

The graduated scale and/or other features printed on or over the absorbent material can be printed from a U.S. Food and Drug Agency (FDA) approved ink, such as a water soluble food grade ink, a plant based dye or stain, an edible material, etc. Any U.S. FDA approved ink may be used that is not inconsistent with the objectives of the present invention. Exemplary FDA approved inks may include FDA approved toner inks that are non-toxic to humans and FDA 21 C.F.R. compliant. The graduated scale and/or other features can indicate an amount and/or a chemical composition of a bodily fluid, including, but not limited to fluids such as saliva, nasal fluid, breast milk, breast discharge, or vaginal fluid. As noted above, the presence or absence of a chemical or elemental constituent or composition, hormone, etc. may also be determined via one or more reactive features printed on or over the absorbent material.

In some embodiments, the substrate comprises a flexible substrate. In other embodiments, the substrate comprises a rigid or semi-rigid substrate. The substrate may be organic or inorganic. In some embodiments, the substrate comprises paper. In other embodiments, the substrate comprises plastic. The substrate can be solid or porous. The substrate can comprise any material that is not inconsistent with the objectives of the instant disclosure.

In some embodiments, the substrate is two-dimensional. In other embodiments, the substrate is three-dimensional. In some embodiments, the substrate has a square, circular, or rectangular sectional shape. The substrate may be planar, non-planar, rounded, angled, or curved. Any size and/or shape of substrate can be provided, so long that it is not inconsistent with the objectives of the instant disclosure.

In some embodiments, the substrate comprises a body portion and a stem portion. A plurality of bendable tabs can extend from portions of the substrate. The tabs are configured to prevent a portion of the device from extending into a body or orifice of a subject and/or prevent a portion of the device from contacting the bodily fluid. For example, in some embodiments, the tabs prevent the stem portion from extending into portions of the subject's body (e.g., the nose, mouth, vagina, etc., of the subject). The tabs are also helpful for elevating the substrate on a flat surface for obtaining easier readings.

In some embodiments, the substrate comprises or is formed from a food approved or food safe material, and/or comprises or is formed from a material which minimizes or reduces the risk of injury to the patient's body.

In some embodiments, the absorbent material is attached to the substrate. For example, the absorbent material may be laminated to the substrate. In some embodiments, the absorbent material is applied via mechanical clamping or heat. Other attachment methods are contemplated.

The substrate can have any dimensions or shape not inconsistent with the objectives of the present invention. For example, in some embodiments, one or more portions of the substrate can be square, rectangular, rounded, elongated, and/or comprise one or more curved sections. Such shapes may be provided in a “one size fits all” shape or size and/or optionally adapted from the “one size fits all” shape or size to a customized size or shape so that subjects of most or all ages and/or sizes may use the devices described herein without modification.

Bodily fluid indicator devices described herein may optionally be provided in a kit. For example, such devices may be provided in kits for use in the measurement of saliva production. Such kits, in some embodiments, comprise a plurality of saliva test members and a display rack. The test members can each comprise a rigid or semi-rigid substrate as described hereinabove.

The display rack for kits described herein may be adapted to retain the saliva test members in a manner permitting visual inspection of the absorbent material of the test members. In certain embodiments, the kits described herein comprise an optional display rack. The display rack can have any architecture or structural features in order to enable the rack to permit visual inspection of the absorbent material of the saliva test members. In some embodiments, the display rack is capable of retaining one or more test members concurrently, permitting a subject or clinician to visually inspect multiple test members simultaneously. The display rack can comprise or be formed from any material. For example, a display rack can comprise or be formed from a plastic, or can comprise or be formed from cardboard or a reinforced paper product. In some embodiments, a container for the saliva test members may also be used as a display rack of a kit described herein. Persons having skill in the art will recognize and appreciate that devices may be provided and used alone, or multiple devices may be provided in a kit for comparison, where desired.

### II. Methods of Using a Bodily Fluid Indicator Device

In a further aspect, methods of using a bodily fluid indicator device are provided. In some embodiments, a method includes providing a bodily fluid indicator device comprising a region of absorbent material and a graduated scale printed thereon. The graduated scale may be printed from a food grade or FDA approved ink that is water soluble.

In some embodiments, the method can further include contacting a bodily fluid with the indicator device for a period of time sufficient to cause the bodily fluid to migrate over a portion of the graduated scale. Notably, the period of time is less than two minutes. In some embodiments, the period of time is approximately one minute or less, approximately 45 seconds or less, between about 30 seconds and two minutes, or a subrange thereof (e.g., 30-45 seconds, 1-2 minutes, etc.). In further embodiments, the period of time is approximately 30 seconds or less.

In some embodiments, the method includes determining an increase or decrease regarding an amount of bodily fluid present based on a proportion of the graduated scale over which the bodily fluid has migrated. The change (i.e., increase or decrease in bodily fluid) may be observed on the amount of the graduated scale over which the bodily fluid has migrated. The subject may then undergo additional testing for diagnosing the cause of a deficient amount of fluid.

In other embodiments, where bodily fluid migrates over three or more portions (e.g., increments) of the graduated scale, the test may indicate that the amount of bodily fluid obtained from a subject is within a normal range. Thus, bodily fluid indicator devices herein can indicate whether a subject's bodily fluid is within a normal range, or a range outside of what is considered normal. What is considered “normal” may vary based on the type of bodily fluid being obtained or the location of the test. For example, increments on a graduated scale for measuring saliva may be appropriate if spaced approximately 5 mm apart whereas increments on a graduated scale for measuring vaginal fluid may be appropriate if spaced approximately 10 mm apart.

As persons having skill in the art will recognize and appreciate, the length, placement, and/or incremental measurements on the graduated scale may be increased (e.g., in length or spacing) or decreased depending on the area being sampled and/or bodily fluid being sampled. Different bodily fluids may be tested using different scales and/or increments. In some methods, determining whether an amount of bodily fluid being produced is normal or abnormal is indicated using a graduated scale. The overall length of the scale and/or the spacing between increments on the scale can be set to an appropriate value for the type of bodily fluid being obtained and/or the area from which the bodily fluid is being obtained.

In some embodiments, methods herein further comprise folding or bending a plurality of tabs disposed on the bodily fluid indicator device for preventing a portion of the device from contacting the bodily fluid. For example, one or more tabs may be folded or bent to provide a “stop” to prevent the entire testing device from being inserted within a subject or otherwise contacting the bodily fluid. In an exemplary embodiment, the tabs are configured to contact a subject's skin or lips to prevent a stem portion of the device from extending within the subject's mouth during a saliva test. Other tabs, for example, may prevent the device from over-extension within a subject's nose, or vagina. Methods herein can be used to test any bodily fluid not inconsistent with the objectives noted herein.

In some embodiments, the device may be devoid of a graduated scale and/or include a graduated scale and one or more other reactive, indicative features printed thereon. Such features may react to a certain attributes of the bodily fluid, for example, temperature, pH, Biomarker, or chemical composition of the bodily fluid. That is, in some embodiments, methods herein can further comprise indicating an absence or presence of a chemical constituent in a bodily fluid based on the reaction (e.g., changes in color, intensity, etc.) of the reactive features over which the bodily fluid has migrated.

For example, such reactive features may indicate the absence or presence of a chemical or elemental constituent (e.g., the presence or absence of iron, calcium, glucose, sucrose, etc.), the absence or presence of a hormone, or the absence of presence of any other chemical attribute that is not inconsistent with the objectives of the instant disclosure. The features may indicate the absence or presence of a chemical or hormone and/or indicate a temperature or pH by changing color, intensity, quantity, size, visibility, etc.

In some embodiments, visibility or non-visibility of one or more reactive features indicates the presence or absence of a chemical or elemental constituent in the bodily fluid. In an exemplary embodiment, a communication feature may be embedded on the device when specified compounds or Biomarkers are detected. Similarly, a feature may be visible on the device when estrogen is detected and invisible when estrogen is not detected. Test devices described herein may be printed with any suitable graduated scale and/or reactive feature for indicating various attributes (e.g., amount, specific elements present, specific compositional constituents present, temperature, pH, hormonal constituents, etc.) relating to a bodily fluid being tested.

In optional embodiments, the saliva test members (i.e., devices) may be weighed before and after insertion into the subject's mouth to obtain an initial weight and a final weight. A first test value can correspond to a weight of saliva present in or on a first saliva test member after use. To obtain a quantitative measurement of salivary production or flow, the first test value can be compared to provide a numerical difference between it and subsequent test values. In certain instances, the first test value can itself be used as a quantitative diagnostic measurement, such as by comparing the first test value to a “standard” value or range of values corresponding to normal, healthy, abnormal, or unhealthy levels of salivary production or flow. Other methods of quantitative measurement or comparison are also possible. For example, one or more of the saliva test members can comprise or include a capillary stem which provides a measurable distance that the saliva travels along the absorbent material in order to obtain a relative quantity of saliva collected on the first saliva test member. This would then be compared to a standard device, which provides a measurable value for comparison and quantification.

Some embodiments described herein are further illustrated in the following non-limiting examples.

### Example 1

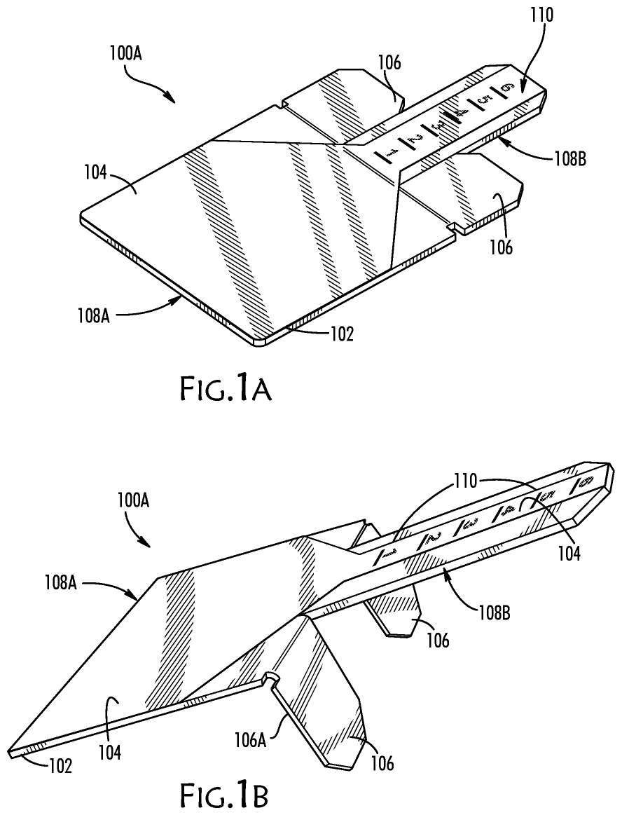

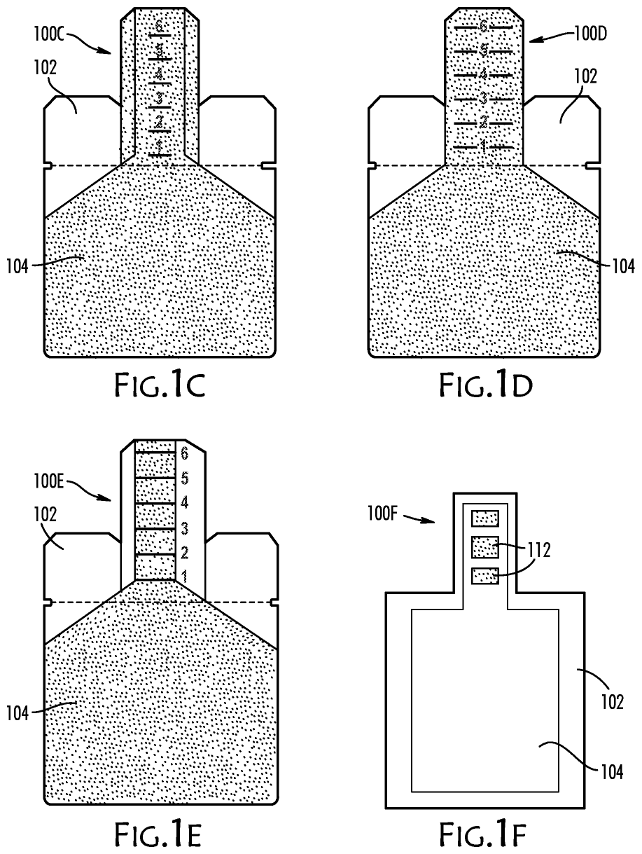

Devices and methods herein are configured to indicate at least an amount and/or a composition a bodily fluid present on an area of a subject or patient. FIGS. 1A and 1B illustrate bodily fluid indicator devices generally designated 100A.

As FIGS. 1A and 1B illustrate, device 100 can include a substrate 102 and an absorbent material 104 disposed on or over a portion of the substrate. Substrate 102 and/or absorbent material 104 can comprise semi-rigid or flexible, non-toxic materials suitable for placement on, over, or within a subject's body. In some embodiments, substrate 102 can comprise a body portion 108A and a stem portion 108B.

In some embodiments, absorbent material 104 comprises a paper, such as a porous filter paper over which a fluid migrates via capillary or osmotic phenomena. Absorbent material 104 is configured to face a mucus membrane or other body portion of a subject that emits or secretes bodily fluid. In an exemplary embodiment, absorbent material 104 is configured to face, contact, and then rest on a subject's tongue during a test. Saliva from the dorsal surface of the tongue contains saliva from all over the mouth (i.e., not just the floor of the mouth where the more powerful salivary glands are located). That is, device 100A is configured to test saliva secreted from multiple salivary glands, including the parotid gland.

Substrate 102 can comprise one or more flexible, creased, or otherwise bendable tabs 106 that can be folded or bent (FIG. 1B) for placement against a subject's skin. In some embodiments, tabs 106 are configured to rest or “stop” against a subject's skin for preventing portions of the device from becoming inserted within the subject's body. For example, in an exemplary embodiment a rear portion 106A of each tab 106 is configured to rest against a subject's lips, while absorbent material 104 disposed on body portion 108A of the substrate 102 rests over the subject's tongue for preventing stem 108B from entering the subject's mouth during the test. During the test, bodily fluid will migrate over a graduated scale 110 printed on device 100A. Graduated scale 110 is shown in broken lines as it is optional and/or may vary in size and/or increments, where desired. Device 100 takes advantage of a natural reflex of the tongue to push saliva to the front of the palate when the mouth closes, thus, saliva can be collected from glands above the tongue and below the tongue for ensuring a more thorough test.

In some embodiments, body portion 108A is at least 0.5 inches wide. In further embodiments, body portion 108A is approximately 1.5 inches wide. In some embodiments, body portion 108A can comprise a width ranging between approximately 0.5-2.5 inches.

In some embodiments, stem portion 108B is at least 0.125 inches wide. In further embodiments, stem portion 108B is approximately 0.25 inches wide. In further embodiments, stem portion 108B is approximately 0.5 inches wide. In some embodiments, stem portion 108B can comprise a width ranging between approximately 0.125-0.75 inches. Stem portion 108A can comprise a length of at least 1 inch or more, 2 inches or more, or a length ranging between approximately 0.5-12 inches.

FIGS. 1C-1E illustrate further example embodiments of bodily fluid indicator devices, generally designated 100C to 100E respectively. Each device 100C to 100E can vary regarding the size, shape, and/or placement of graduated scale 110 and/or the size, shape, and/or placement of absorbent material 104. Such embodiments are shown for illustration purposes only. Persons having skill in the art will recognize and appreciate that devices may look a little different in regards to the sizes, shapes, and/or placement of various features.

FIG. 1F is an example embodiment of a bodily fluid indicator device 100F that is devoid of a graduated scale. However, FIG. 1F illustrates one or more reactive features 112 printed thereon. Such features 112 may be reactive (e.g., change in color, size, shape, intensity, etc.) to various chemicals, elements, hormones, bacteria, yeast, fluid temperature, fluid pH, etc., for indicating the absence or presence of a chemical or elemental constituent and/or a chemical composition forming the bodily fluid. Features 112 can indicate any number of chemical or physical attributes regarding the bodily fluid that migrates across the features.

For example, features 112 may react (e.g., change in size, shape, color, intensity, visibility, etc.) to calcium for indicating an amount of calcium present in the bodily fluid. In a further exemplary embodiment, features 112 may target and react to a specific hormone for indicating the presence or absence of the hormone in the bodily fluid.

Notably, devices described herein may include a graduated scale (e.g., 110), reactive features (e.g., 112), or a combination of both for indicating whether the type and/or amount of bodily fluid being produced is within a measurable range. As bodily fluid migrates over the graduated scale 110 and/or features 112, various attributes (e.g., chemical attributes, temperature, pH, volume, amount, production, gland viability, etc.) are indicated and become apparent.

### Example 2

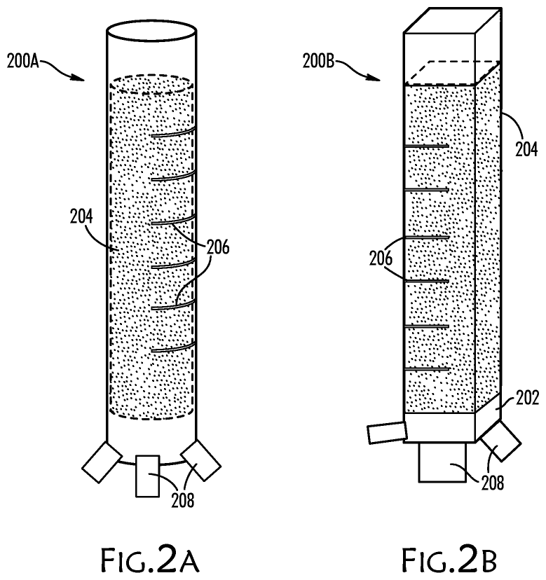

FIGS. 2A-2B are various schematic illustrations of bodily fluid indicator devices generally designated 200A-200B, respectively. FIG. 2A is an example device 200A comprising a curved or rounded multidimensional substrate 202 and a correspondingly shaped region or area of absorbent material 204. Increments 206 forming a graduated scale are provided (e.g., printed) over the absorbent material. A plurality of tabs 208 can be disposed at one end for preventing the entire device from extending within a cavity of a subject's body. For example, and in some embodiments, the tabs 208 may prevent the device from fully extending within a subject's nasal passage or vagina. Substrate 202 may be solid or hollow, where desired.

FIG. 2B is an example device 200B comprising an angular, multidimensional substrate 202 and a correspondingly shaped region or area of absorbent material 204. Increments 206 forming a graduated scale are provided (e.g., printed) over the absorbent material. A plurality of tabs 208 can be disposed at one end of device 200B for preventing the device from extending within a cavity of a subject's body.

### Example 3

FIG. 3 is an example embodiment of a method, generally designated 300, of using a bodily fluid indicator device to indicate an amount and/or composition of a bodily fluid. In block 302, a bodily fluid indicator device is provided. The device comprises a region of absorbent material and a graduated scale printed thereon. The graduated scale may be printed using a water soluble food grade ink. In lieu of and/or in addition to a graduated scale, one or more other features or shapes may also be printed on the absorbent material (see e.g., FIG. 1F). The features may indicate the absence or presence of a chemical or elemental constituent and/or a component of the bodily fluid.

In block 304, the device contacts a bodily fluid for a period of time sufficient to cause the bodily fluid to migrate over a portion of the graduated scale. Notably, the period of time can be less than two minutes or less than one minute.

In block 306, the measurable amount of bodily fluid is determined based on a proportion of the graduated scale over which the bodily fluid has migrated.

In another exemplary embodiment, a method of using a bodily fluid indicator device is provided. The method includes providing a bodily fluid indicator device comprising a region of absorbent material and one or more features printed thereon.

The method can further comprise contacting a bodily fluid with the indicator device for a period of time sufficient to cause the bodily fluid to migrate over a portion of the one or more features, wherein the period of time is less than one minute.

The method can further comprise indicating an absence or presence of a chemical constituent forming the bodily fluid based on a property or attribute regarding the one or more features over which the bodily fluid has migrated. For example, the features may indicate the absence or presence of a chemical or elemental constituent (e.g., the presence or absence of iron, calcium, glucose, sucrose, etc.), the absence or presence of a hormone, or the absence of presence of any other chemical attribute that is not inconsistent with the objectives of the instant disclosure. The one or more properties or attribute may include a color, intensity, or size of the one or more features for indicating the presence or absence of a chemical or elemental constituent in the bodily fluid.

In other example embodiments according to aspects of the present technology, a bodily fluid indicator is provided comprising a substrate (for example having a body portion and a stem portion) having at least a first surface and a second surface. At least a portion or a plurality of portions of at least one of the surfaces is covered with or comprises an absorbent material, and another portion or plurality of other portions are devoid of any absorbent material. In some instances, a graduated scale or one or more reactive features is printed on the absorbent material. Such reactive features may be reactive (e.g., change in color, size, shape, intensity, etc.) to various chemicals, elements, hormones, bacteria, yeast, fluid temperature, fluid pH, etc., for indicating the absence or presence of a chemical or elemental constituent and/or a chemical composition forming the bodily fluid. Such features can further indicate any number of chemical or physical attributes regarding the bodily fluid that migrates across the features. According to some embodiments, the absorbent material may be embedded with nanoparticles and/or nanoparticle reactive agents which are capable of measuring an aspect of collected bodily fluid. In some instances, the absorbent material is an organic material, such as a cellulose based material, and in some other instances, the absorbent material is an inorganic material. In some embodiments, the bodily fluid indicator device can incorporate attached or printed features on the non-absorbent material portions capable of producing a detection and/or measurement signal such as a colorimetric or electronic signal to indicate the presence of pathogens, viruses, enzymes, hormones, metabolites, metals or microbial contaminates. In some embodiments, the bodily fluid indicator device can include one or more biosensors and readers.

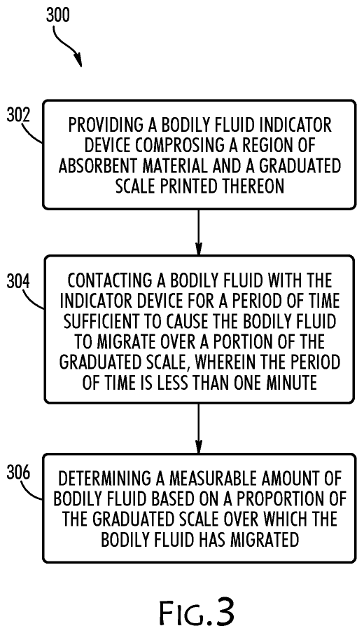

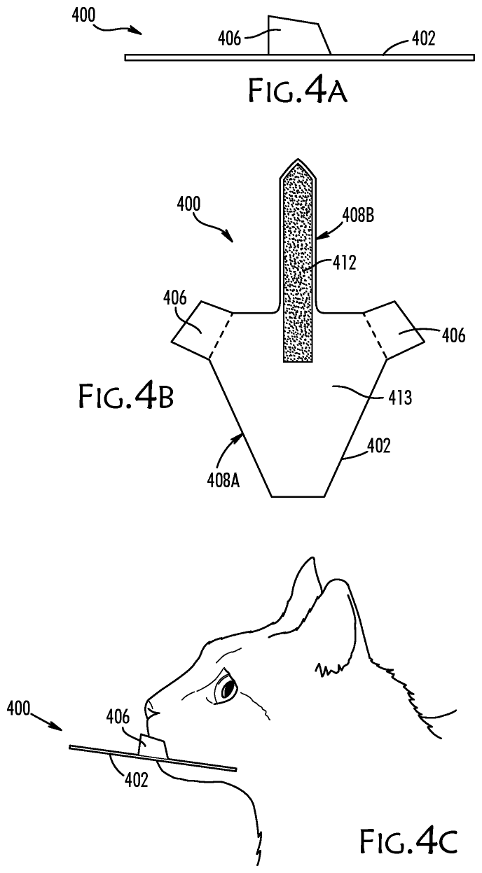

The bodily fluid indicator device can include one, two, or more bendable tabs which can be configured, as well as with the shape or geometry of the substrate, fit or otherwise accommodate a given subject for bodily fluid collection and/or measurement. The one or more bendable tabs can extend from the substrate, or more specifically from a substrate body portion. In some example embodiments, a bodily fluid indicator device can be implemented to collect bodily fluid according to methods described herein from a domestic mammal, such as a cat, dog, horse, bovine, swine, among others. It will be appreciated that the shape and/or geometry of the substrate and the one or more bendable tabs that extend from the substrate body can be configured to be consistent with the size of a domestic animal on which the bodily fluid indicator device is used. Accordingly, the configuration of a bodily fluid indicator device and/or portions or aspects thereof may be elongated, shortened, widened, angled, rounded or undergo any other geometric transformations to accommodate a species of interest (e.g. bodily fluid indicator device 400 of FIG. 4C).

The bodily fluid indicator device can be configured and implemented to collect and evaluate bodily fluid from a subject, such as a domestic mammal. Bodily fluid can be collected by the bodily fluid indicator device and the collected bodily fluid can be evaluated via or on the surface of the absorbent material. The bodily fluid collected can be tested via the bodily fluid indicator device to determine the presence of, for example, pathogens, viruses, enzymes, hormones, metabolites, metals, and/or microbial contamination. In some other instances, the bodily fluid collected can be tested via the bodily fluid indicator device to determine the presence of pharmaceutical contamination. According to various embodiments, a bodily fluid indicator device can comprise a substrate (the substrate may incorporate a body portion, a stem portion, and one or more placement tabs, among other features) that has some areas or portions thereof comprising or covered with an absorbent material and at least another portion that is devoid of absorbent material and will have attached and/or printed features that can produce a signal (e.g. colorimetric, electronic, etc.) to indicate the presence of for example, pathogens, viruses, enzymes, hormones, metabolites, metals or microbial contaminates in collected bodily fluid(s). In some embodiments, the bodily fluid indicator device can detect and/or measure one or more of five pathogens, viruses, enzymes, hormones, metabolites, metals or microbial contaminates simultaneously.

In some embodiments, bodily fluid collected by a bodily fluid indicator device according to aspects described herein can be collected on a surface of the device and exposed to testing materials. In some instances, bodily fluid collected on a bodily fluid indicator device can further be capable of visible change under natural, fluorescent, and/or UV illumination.

It will be appreciated that the incorporation of sensitive detection materials, such as nanomaterials, into the bodily fluid indicator device will obviate the need for laboratory interpretation and as such measurements can be directly taken and interpreted by the bodily fluid indicator device. Example advantages include, but are not limited to the device being a lightweight, portable screening device easily incorporated into emergency or routine use for swift screening for pathogens, viruses, enzymes, hormones, metabolites, metals or microbial contaminates.

According to various embodiments, a bodily fluid indicator device can be utilized to collect and measure aspects of bodily fluid from a human and/or animal. Bodily fluid may be collected from a subject that is living or from a subject that is not living or recently deceased. Examples of bodily fluids that may be collected or absorbed by the bodily fluid indicator device include, but are not limited to, saliva, sweat, urine, nasal fluid, breast milk, breast discharge, vaginal fluid, rectal fluid, among others. In some embodiments, the bodily fluid collected may be from the central nervous system of a human or animal.

Referring now to FIG. 4A-4C, bodily fluid indicator devices 400 are illustrated that can be configured to collect bodily fluid from a human or an animal and indicate the presence, amount, and/or composition of a bodily fluid present with respect to a subject. Devices 400 can include a substrate 402 and an absorbent material disposed on or over a portion of the substrate. In some instances, portions of the substrate comprise the absorbent material. In some embodiments, substrate 402 can comprise a body portion 408a and a stem portion 408b. Substrate 402 can comprise one or more flexible, creased, or otherwise bendable tabs 406 that can be folded or bent to control placement of the device with respect to a subject. Referring briefly to FIG. 4C, device 400 has tabs 406 which control placement of the device for saliva collection in a domestic animal, for example. It will be appreciated that the shape of the substrate and the plurality of bendable tabs extending from the surface are consistent with the size of the domestic animal and that the configuration may be elongated, shortened or broadened to accommodate the species of interest (e.g. shown in FIGS. 4A-4C). As an example here, in the configuration illustrated in FIGS. 4A-4C, the stem is lengthened, the tabs are placed at angles to safeguard the fingers of the handler and prevent swallowing by the animal. Further, the geometry of the substrate body is typically angled to better accommodate the species of interest (e.g. domestic animals, such as household animals or farm animals, or wild animals).

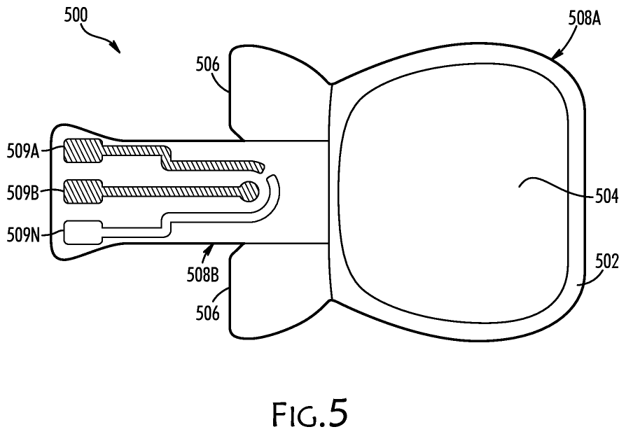

Turning to FIG. 5, an example body fluid indicator device 500 is illustrated in accordance with some embodiments of the present technology. Device 500 can include, among other features not shown, substrate 502 and absorbent material 504 on or over a portion of the substrate. Substrate 502 can comprise body portion 508a, stem portion 508b, and tabs 506. Device 500 can further include one or more sensors, biosensors (e.g. magnetic microbeads, among others), electrodes, and/or substances (e.g. chemically and/or biologically reactive substances) 509a, 509b, 509n that are configured to produce a signal, e.g. colorimetric and/or electronic to indicate and/or in response to the presence of pathogens, viruses, enzymes, hormones, metabolites, metals, or microbial contaminates in collected bodily fluid, for example by absorbent material 504. The sensors, biosensors, electrodes, and/or substances 509a, 509b, 509n can be incorporated into the material or printed thereon, for instance by jet printing or additive manufacturing.

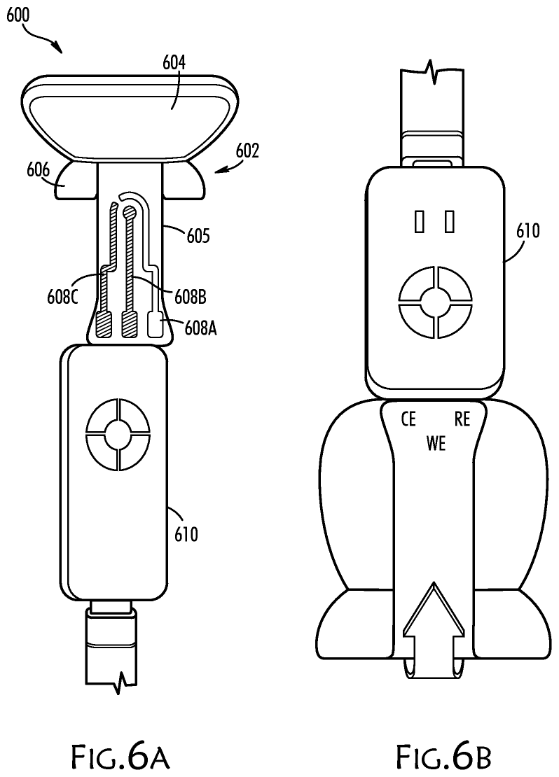

Turning briefly to FIGS. 6A and 6B, the body fluid indicator device described above and including one or more sensors, biosensors, electrodes, and/or substances (e.g. reactive substances) may be used in conjunction with a portable reader that can be connected to a portable analysis device such as a potentiostat which can work in conjunction with the electrodes and/or sensors and/or reactive substance(s) of the bodily fluid indicator device to detect and/or otherwise analyze the presence of pathogens, viruses, enzymes, hormones, metabolites, metals or microbial contaminates in bodily fluid collected. In other instances, the absence or presence of a constituent (e.g. biological or chemical) of a collected bodily fluid sample and/or an amount of a bodily fluid sample. In some instances, the rate at which the sample is being collected (or the rate at which the fluid is being produced) can be determined. Looking more particularly at FIG. 6A a bodily fluid collection and analysis or characterization system is illustrated. Bodily fluid collection and analysis system 600 can include a bodily fluid device 602, which can comprise a body portion 604, one or more tabs 606, and a stem portion 605. In some aspects an absorbent material may be disposed over the body portion and/or over the stem portion. In one example, absorbent material (e.g. an absorbent pad) is disposed over a portion of the body portion 604. Bodily fluid device 602 can further incorporate one or more sensors, biosensors, electrodes, and/or substances or reactive substances 608a, 608b, 608c that can be configured to produce a signal, e.g. colorimetric, chemical, and/or electronic signal to indicate and/or in response to the presence of a component of the bodily fluid collected. As will be appreciated the one or more sensors, biosensors (e.g. magnetic microbeads), electrodes, and/or substances or reactive substances 608a, 608b, 608c are configured to be in operable communication with the absorbent material of the bodily fluid device 604. System 600 can further include one or more sample measurement and/or characterization devices, for example an electrode holder connected with a portable potentiostat 610.

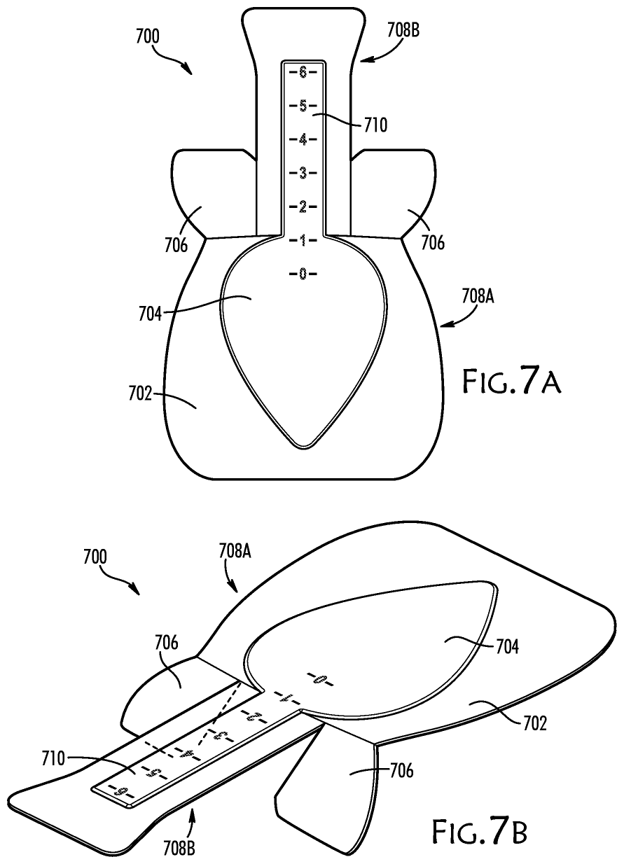

Referring to FIGS. 7A and 7B, example bodily fluid indicator device(s) 700 are illustrated, in accordance with aspects of the present technology. As with be appreciated, a bodily fluid indicator device 700 can be configured and/or used to collect bodily fluid from a human or an animal and further indicate the presence, amount, and/or composition of a bodily fluid present or collected with respect to a subject. Device(s) 700, e.g. human and/or animal bodily fluid collection, measurement, and/or characterization device can include a substrate 702 and an absorbent material 704 disposed on or over a portion of the substrate. According to some aspects, substrate 702 comprises a body portion 708a and a stem portion 708b. Substrate 708, body portion 708a, and/or stem portion 708b can be configured in any shape not inconsistent with the objectives of this disclosure. For instance, any of the substrate 708, body portion 708a, and/or stem portion 708b can have a plurality of various edges, angular sections or edges, and/or rounded sections or edges. For example, body portion 708a can incorporate rounded edges and/or corners and stem portion 708b can incorporate rounded edges and/or corners, or further for example a flared end portion that can be configured to enable a more precise and/or accurate use of the device. Device 700 can further include one or more tabs 706 which can be configured to control the placement of a device during the collection of bodily fluid(s) of a subject. One or more tabs 706 can be flexible, creased, or otherwise bendable such that each tab can be folded or bent. As will be appreciated, each tab can independently be folded or bent to a requisite degree depending on a subject (e.g. human versus animal) or application (e.g. such as based on a location of where a bodily fluid may be collected). It will be appreciated that the shape of the substrate and/or its components and the plurality of bendable tabs extending from the surface of the substrate can be configured based on the subject dimensions and the location of where a sample may be taken, and that the configuration of each of the elements of the device may be elongated, shortened, broadened, angled, etc. to accommodate the species of interest. Absorbent material 704 can also have a shape not inconsistent with the objectives of the present disclosure. For example, absorbent material 704 may be configured to fit the shape of the substrate 702. Alternatively, or additionally, absorbent material 704 can be shaped based on the subject or application for which it is being used to most effectively or efficiently collect a sample or, for instance, for ease of placement. Device 700 can also include one or more components for the collection and characterization of bodily fluid. In some aspects, device 700 can include a graduated scale 710, which may be integrated with absorbent material 704. In some other aspects, graduated scale 710 (or other component) may be separate from the absorbent material disposed on the body portion 708a but in communication with the absorbent material 704, e.g. via a connection component.

It will be appreciated that device 700 can have additional sample collection and characterization features, such as reactive features or a combination of both for indicating whether the type and/or amount of bodily fluid being produced is within a measurable range. As previously describes as bodily fluid migrates over the graduated scale 710 and/or other features during collection, various attributes (e.g., chemical attributes, temperature, pH, volume, amount, production, gland viability, etc.) can be determined as a part of the characterization of the bodily fluid being collected.

According to embodiments of the present technology, a bodily fluid collection, indicator, and/or characterization device (also referred to as a bodily fluid device) is provided. The device can comprise a substrate (e.g. a curved substrate or a planar substrate) comprising one or more surfaces and an absorbent material that covers at least a portion of a surface of the substrate. In some instances, a substrate can be flexible, and in others, a substrate can be semi-flexible or rigid. As will be appreciated, absorbent material can be positioned on any surface of the substrate. As previously described, a substrate can include a body portion and a stem portion. A bodily fluid device can further comprise one or more bendable tabs that extend from the substrate, for example extending from the body portion of the substrate. As will be appreciated, the one or more bendable tabs can be movable between at least a first position and at least a second position. In some instances, a first position can be coplanar with the substrate and the second position is non-coplanar with the substrate. In some instances, one or more bendable tabs can be moved to any angle relative to the substrate and/or the body portion and/or the stem portion. For example, one or more bendable tabs can be moved anywhere between from 0°-45°, from 0°-90°, from 0°-135°, and/or from 0°-180°. According to some further aspects, an absorbent material can comprise a plurality of portions or zones. For example, a first portion of the absorbent material is configured to contact a source of bodily fluid of a subject and allow for the bodily fluid to migrate towards at least a second (or third, or fourth, etc.) portion of the absorbent material. In some aspects, the second portions (or additional portions) can comprise one or more fluid characterization features, such as a graduated scale, a reactive feature, a sensor, a biosensor, an electrode, and/or other substance that can be incorporated into and/or printed on the substrate, for instance on one or more of a body portion and a stem portion.

Referring back to the one or more bendable tabs, the tabs can extend from any position on the substrate, and further can extend from any position relative to the absorbent material disposed on the substrate. For example, one or more tabs can extend from a location on the substrate that is between a first end (or portion) and a second end (or portion) of the absorbent material. As will be appreciated, the tabs can be adjusted or bent or configured to prevent at least one of the first end (or portion) or the second end (or portion) of the absorbent material from contacting the source of bodily fluid during collection of a sample.

In some further aspects, a bodily fluid device can incorporate one or more sample characterization features. In some instances, a sample characterization feature can a graduated scale, a reactive feature, a sensor, a biosensor, an electrode, and/or other substance that can be incorporated into and/or printed on the substrate and/or the absorbent material. In some aspects, a graduated scale can be configured to determine an amount of bodily fluid produced by a subject based on a proportion of the graduated scale over which the bodily fluid has migrated or traveled during collection, for instance during a period of time. According to some other aspects, a reactive feature can be configured to determine an absence or presence of a chemical constituent of bodily fluid collected based on a reaction indicated or signaled by the reactive feature over which a bodily fluid sample has migrated or traveled.

In other embodiments, a method of using a bodily fluid device is provided, the method may comprise providing a bodily fluid device (e.g. bodily fluid indicator device) and contacting, in some instances through flexing, a bodily fluid of or from a subject with at least a portion of the bodily fluid device (e.g. an absorbent material disposed on a portion of a substrate of the device) for a period of time (e.g. a pre-determined period of time or a known period of time) that is sufficient to allow the bodily fluid to migrate or travel from a first portion of absorbent material to a second portion of absorbent material. Based on the migration of bodily fluid, a method can further comprise determining at least one of an amount of bodily fluid being produced (e.g. a measureable amount of bodily fluid being produced by a subject) and/or the presence or absence of a chemical constituent of the bodily fluid. In some instances, bodily fluid is contacted with the bodily fluid device for less than 1 minute, for less than 3 minutes, for less than 5 minutes, or for less than 10 minutes, up to one minute, up to 3 minutes, up to 5 minutes, or up to 10 minutes. During the contact period bodily fluid can migrate from a first portion of absorbent material to at least one of a second portion of absorbent material, over a portion of a graduated scale, or over a portion of a reactive feature, a sensor and/or a biosensor, an electrode and/or a light source. Based on the migration of bodily collected in a sample period an amount of fluid and/or an absence or presence of a chemical constituent of the fluid can be determined.

Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below. Certain features and subcombinations are of utility and can be employed without reference to other features and subcombinations and are contemplated within the scope of the claims.

1. A bodily fluid indicator device comprising:
a substrate; and
an absorbent material covering a portion of the substrate, wherein the absorbent material comprises at least one of a graduated scale and/or a reactive feature.

2. The device of claim 1, wherein the absorbent material is configured to collect and evaluate bodily fluid of a subject.

3. The device of claim 1, wherein the bodily fluid indicator device is configured to determine the presence of at least one of pathogens, viruses, enzymes, hormones, metabolites, metals, or microbial contamination in collected bodily fluid of a subject.

4. The device of claim 1, wherein the presence of a pharmaceutical contamination is detected in collected bodily fluid of a subject.

5. The device of claim 1, wherein the device is configured to collect bodily fluid from a domestic animal.

6. The device of claim 1, further comprising one or more bendable tabs configured for placement of the device with respect to a subject.

7. The device of claim 1, wherein collected bodily fluid of a subject on the absorbent material is exposed to one or more testing materials.

8. The device of claim 1, wherein collected bodily fluid of a subject on the absorbent material exhibits a visible change when exposed to natural, fluorescent, and/or UV illumination.

9. The device of claim 1, wherein bodily fluid of a subject is collected from a subject that is not living and/or recently deceased.

10. The device of claim 1, wherein the bodily fluid of a subject is collected from the central nervous system of the subject.

11. The device of claim 1, wherein the absorbent material comprises one or more nanoparticle reactive agents.

12. The device of claim 1, wherein the absorbent material comprises a cellulose-based material.

13. The device of claim 1, wherein the absorbent material comprises an inorganic material.

14. The device of claim 1, configured to collect bodily fluid comprising at least one of saliva, sweat, urine, nasal fluid, breast milk, breast discharge, vaginal fluid, and/or rectal fluid.

15. The device of claim 1, wherein the substrate further comprises at least one of a biosensor, sensor, and/or electrode.

16. The device of claim 15, wherein the at least one biosensor, sensor, and/or electrode is in operable communication with the absorbent material.

17. The device of claim 16, wherein the at least one biosensor, sensor, and/or electrode is configured to produce a signal that is colorimetric and/or electronic.

18. The device of claim 17, wherein the signal corresponds to an indication of the presence of at least one of pathogens, viruses, enzymes, hormones, metabolites, metals or microbial contaminates.

19. The device claim 17, wherein the device is configured to produce a plurality of signals corresponding to a plurality of pathogens, viruses, enzymes, hormones, metabolites, metals or microbial contaminates.

20. The device of claim 15, wherein the at least one of a biosensor, sensor, and/or electrode is attached or printed onto the device.
